(19) United States
(12) Patent Application Publication
(10) Pub. No.: US 2024/0115564 A1
(43) Pub. Date: Apr. 11, 2024

(54) HTR1F ANTAGONISTS FOR IMPROVEMENT OF BETA CELL SURVIVAL AND FUNCTION

(71) Applicant: The Regents of the University of California, Oakland, CA (US)

(72) Inventor: Gregory Ku, San Francisco, CA (US)

(73) Assignee: The Regents of the University of California, Oakland, CA (US)

(21) Appl. No.: 18/271,588

(22) PCT Filed: Jan. 11, 2022
(86) PCT No.: PCT/US2022/012032

§ 371 Date: Jul. 10, 2023

Related U.S. Application Data
(60) Provisional application No. 63/136,434, filed on Jan. 12, 2021.

(51) Int. Cl. A61K 31/496; A61K 31/4709; A61K 31/48; A61K 31/7088; A61P 3/10

(52) U.S. Cl. CPC A61K 31/496; A61K 31/4709; A61K 31/48; A61K 31/7088; A61P 3/10; A61K 2039/505

(57) ABSTRACT

Methods for treating diabetes are described. The methods include administration of a serotonin receptor 1F (HTR1F) antagonist, such as a substituted piperidine, methysergide, or methiothepin, to a subject in need thereof. Administration of the HTR1F antagonist can increase survival of pancreatic beta cells in conjunction with pancreatic islet transplantation. Methods for transplanting pancreatic islets to subjects such as diabetes patients are also described.

### CROSS-REFERENCES TO RELATED APPLICATIONS

The present application is a U.S. 371 National Phase patent application of International Application No. PCT/US2022/012032 filed Jan. 11, 2022, which claims priority to U.S. Provisional Pat. Appl. No. 63/136,434, filed on Jan. 12, 2021, which applications are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jul. 10, 2023, is named 081906-241010PC-1289217_SL.txt and is 1,674 bytes in size.

### BACKGROUND OF THE INVENTION

Type 1 diabetes is caused by an autoimmune attack on the pancreatic beta cells. The cure for type 1 diabetes must involve replacing or regenerating these critical cells (likely combined with immunosuppression). Indeed, type 1 diabetes can be cured by islet transplantation but many beta cells die soon after they are infused into the portal vein, meaning that only recipients with low insulin requirements can be cured with islets from a single donor [1, 2]. Therefore, new therapeutics that improve human beta cell survival are expected to make islet transplantation a possibility for more type 1 patients. While ES-cell derived beta cells may soon allow a virtually unlimited supply of beta-like cells, these cells also die in significant numbers upon transplant [3]. Outside of the transplant setting, beta cell death is an important part of the pathophysiology of both type 1 and type 2 diabetes [4] and understanding human beta cell death is expected to reveal new targets to treat all forms of diabetes.

Increasing human beta cell replication would be a complementary approach to reducing beta cell death. Many studies of beta cell replication have identified candidates in rodent beta cells and then asked if these candidates can drive primary human beta cell replication. Unfortunately, while many new mouse beta cell mitogens have been found, most have proven ineffective in adult human beta cells (recently reviewed by [5]). Starting in rodent cells is not ideal since this strategy cannot identify any biology of primary human beta cells that is not conserved in rodents. Indeed, the biology of the human beta cell can be quite different than that of the mouse or rat, particularly with regard to proliferation (reviewed by [6]). A bright spot in the area of human beta cell replication has come from chemical screens [7-10]. Interestingly, though these studies started with diverse libraries and/or screening strategies, most have converged on the inhibition of a single target, DYRKIA [8, 9, 11, 12].

While the DYRKIA inhibitors have certainly been a game changer, a major gap in the field remains a relative paucity of other pathways that can trigger primary human beta cell replication.

### BRIEF SUMMARY OF THE INVENTION

Provided herein are methods for treating diabetes. The methods include administration of a serotonin receptor 1F (HTR1F) antagonist to a subject in need thereof.

In some embodiments, the HTR1F antagonist is a compound according to Formula I

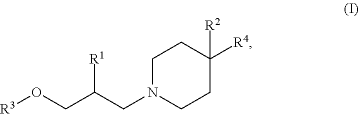

or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

Also provided herein are methods for transplanting pancreatic islets. The methods include delivering pancreatic islets to a subject in conjunction with an HTR1F antagonist

### BRIEF DESCRIPTION OF THE DRAWINGS

FIG. 1A shows the design of a pooled shRNA dropout screen in primary human beta cells. A cell with an shRNA targeting a negative regulator of proliferation or survival is labeled with an arrow at the top of FIG. 1A, and a higher frequency of these cells is shown after transplant.

FIG. 1B shows fold-enrichment after transplant of 25 shRNAs targeting CDKN1B (grey) or 500 non-targeting shRNAs (black). Each line represents a different donor. A total of 3 donors are shown.

FIG. 1C shows fold-enrichment as in FIG. 1B, but for shRNAs targeting HTR1F (grey).

FIG. 1D shows enrichments for each of the HTR1F targeting shRNAs averaged over the 3 donors (grey) and 20 randomly selected negative controls shRNAs (black). Note that these enrichments are represented as the log 2 fold enrichment. Standard error is shown over the 3 independent donors.

FIG. 2A shows islet cell death after treatment with an HTR1F specific agonist. Dissociated human islets were treated with the indicated doses of LY344864 for 24 hours. Dead cells were monitored by Sytox Green. The number of Sytox Green nuclei were normalized to cell density measured by bright field imaging. n=4 biological replicates for each donor. *p<0.05, **p<0.01 by post-hoc Student's t-test after one-way ANOVA versus vehicle treatment.

FIG. 2B shows islet cell death as in FIG. 2A, but cells were treated with the indicated doses of LY344864 for 24 hours and stained by TUNEL and for insulin. A representative image is shown. White arrows represent cells that are TUNEL+ insulin+ cells. Scale bar is 25 μM.

FIG. 2C shows the quantitation of FIG. 2B over 4 independent donors. Given differences in baseline cell death between donors, the fold increase in the fraction of TUNEL+ beta cells over vehicle control is plotted. Standard error is shown, n=4 donors with at least 400 beta cells counted for each donor. *p<0.05, one-way ANOVA, post-hoc tested against vehicle.

FIG. 2D shows cell death in MIN6 cells treated with either vehicle or pertussis toxin and then transiently transfected with either GFP or GFP-T2A-HTR1F. GFP+ cells were assessed for cell death by propidium iodide staining 32-40 hours after transfection. Two-way ANOVA analysis showed an effect of HTR1F expression f(1)=11.9, p=0.000762 and an effect of pertussis toxin treatment f(1)=10.494, p=0.001529, but f(1)=1.969, p=0.162 for interaction. Significant post-hoc Holm adjusted Student's t-test comparisons are shown. n=33 for each group conducted over 3 independent experiments. Taken together, FIGS. 2A-2D illustrate the induction of primary human beta cell death by HTR1F signaling.

FIG. 2E shows the efficient knockdown of HTR1F with an HTR1F shRNA. K562 cells were infected with a lentivirus containing puromycin resistance and either control or HTR1F shRNA, selected with puromycin and RT-QPCR was performed for HTR1F. n=5. *p<0.05 by Student's t-test.

FIG. 3A shows a strategy for measuring beta cell death in the setting of transplant.

FIG. 3B shows beta cell death measured after human islets were infected with control shRNA lentivirus (GFP) or HTR1F shRNA lentivirus (mCherry), transplanted, explanted, and stained for cleaved caspase-3, mCherry, and GFP as depicted in FIG. 3A. The % reduction in cleaved caspase-3 in mCherry positive cells (HTR1F knockdown) as compared to the GFP positive cells (control knockdown) is plotted. n=4 donors. **p<0.01 by one sample, two-tailed Student's t-test. Baseline cell death ranged between 5-10%.

FIG. 3C shows beta cell death in dissociated human islets treated with 10 μM thapsigargan, 30 nM methysergide, or both for 48 hours. % dead cells were monitored by Sytox Green staining at 48 hours and normalized to total nuclei. n=3 biological replicates per condition. Each graph is from an independent donor. *p<0.05 for thapsigargan and for the interaction between methysergide and thapsigargan by 2-way ANOVA. Standard error is shown.

FIG. 3D shows diabetes-free survival in test animals receiving marginal islet mass transplants of human islets treated with vehicle or methysergide. *p<0.05 by log-rank (Mantel-Cox) test. Taken together, FIGS. 3A-3D illustrate the prevention of human beta cell death by blocking of HTR1F signaling.

FIG. 4 shows inhibition of HTR1F in 293T cells by 1-(2-hydroxy-3-(naphthalen-2-yloxy)propyl)-4-(quinolin-3-yl)piperidin-4-ol.

FIG. 5 shows glucose-stimulated insulin release in human islets treated with HTR1F antagonist 1-(2-hydroxy-3-(naphthalen-2-yloxy)propyl)-4-(quinolin-3-yl)piperidin-4-ol (right bars) as compared to control islets (left bars). Antagonist structure is shown above the data plot.

### DETAILED DESCRIPTION OF THE INVENTION

Islet transplantation can cure type 1 diabetes but peri-transplant beta cell death limits this procedure to those with low insulin requirements. Improving human beta cell survival or proliferation may make islet transplantation a possibility for more type 1 patients. The present invention is based, in part, on the identification of regulators of beta cell survival and proliferation using a pooled RNA interference screen in primary human beta cells transplanted into mice. Small hairpin RNAs targeting cyclin dependent kinase inhibitors were enriched after transplant, showing that the screen was able to detect negative regulators of beta cell proliferation. Blocking of signaling mediated by the Gαi-coupled, serotonin 1F receptor (HTR1F) was found to reduce beta cell death, whereas triggering HTR1F signaling induced beta cell death. Methysergide, a serotonin receptor antagonist formerly used for headache prophylaxis, improved glycemia in a mouse model of human islet transplant. Inhibition of HTR1F was also found to increase insulin production in human islets. The studies described herein show that HTR1F is a novel target to improve human beta cell survival during islet transplantation and for the treatment of metabolic disorders such as diabetes.

### I. Definitions

As used herein, the term “diabetes” refers to a variable disorder of carbohydrate metabolism caused by a combination of hereditary and environmental factors. Diabetes includes, but is not limited to, type 1 diabetes, where the pancreas produces little or no insulin; type 2 diabetes, where a subject becomes resistant to insulin or cannot produce sufficient insulin; gestational diabetes; prediabetes; and metabolic syndrome. Diabetes is typically characterized by inadequate secretion or utilization of insulin, by excessive urine production, by excessive amounts of sugar in the blood and urine, and by thirst, hunger, and loss of weight.

As used herein, the terms “serotonin receptor 1F,” “5-hydroxytryptamine receptor 1F,” and “HTR1F” refer to a G-protein coupled receptor for 5-hydroxytryptamine (serotonin). HTR1F is a G_{i/o}-type receptor, and activation leads to inhibition of adenylyl cyclase and decreased production of cAMP. Human HTR1F (GenBank Accession No. AAM21128.1) is expressed in various cells and tissues including, but not limited to, brain tissue; airway epithelial cells; subcutaneous adipose tissue; kidney tissue; and pancreatic beta cells, alpha cells, delta cells, and gamma cells. Over 200 orthologs to human HTR1F have been identified in various species including, but not limited to, mouse (M. musculus), cattle (B. taurus), dog (C. lupus), sheep (O. aries), and chimpanzee (P. troglodytes).

As used herein, the term “pancreatic islet” refers to cell aggregates containing pancreatic endocrine hormone producing cells: alpha cells, beta cells, delta cells, gamma cells epsilon cells, and combinations thereof. In vivo, islets (also referred to as islets of Langerhans) are roughly spherical and distributed throughout the pancreas, surrounded by exocrine tissue. Pancreatic islets may be obtained from a deceased donor and transplanted to a subject as described herein (e.g., to the liver of the transplant recipient).

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups may be substituted with one or more groups selected from halo, hydroxy, amino, oxo, alkylamino, amido, acyl, nitro, cyano, and alkoxy.

As used herein, the term “alkoxy,” by itself or as part of another substituent, refers to a group having the formula —OR, wherein R is alkyl.

As used herein, the term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. For example, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

As used herein, the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. For example, “substituted heterocyclyl” groups can be substituted with one or more groups selected from halo, hydroxy, amino, oxo, alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

As used herein, the terms “halogen” and “halo,” by themselves or as part of another substituent, refer to fluorine, chlorine, bromine and iodine.

As used herein, the term “hydroxy” refers to the moiety —OH.

As used herein, the term “oxo” refers to an oxygen atom that is double-bonded to a compound (i.e., O═).

As used herein, the term “amino” refers to a moiety —NR₂, wherein each R group is H or alkyl. An amino moiety can be ionized to form the corresponding ammonium cation. “Alkylamino” refers to an amino moiety wherein at least one of the R groups is alkyl.

As used herein, the term “amido” refers to a moiety —NRC(O)R or —C(O)NR₂, wherein each R group is H or alkyl.

As used herein, the term “acyl” refers to the moiety —C(O)R, wherein each R group is alkyl.

As used herein, the term “nitro” refers to the moiety —NO₂.

As used herein, the term “cyano” refers to a carbon atom triple-bonded to a nitrogen atom (i.e., the moiety —C≡N).

As used herein, the term “carboxy” refers to the moiety —C(O)OH.

As used herein, the term “salt” refers to a compounds comprising at least one cation (e.g., an organic cation or an inorganic cation) and at least one anion (e.g., an organic anion or an inorganic anion). Acid salts of basic HTR1F antagonists include, but are not limited to, mineral acid salts (e.g., salts formed using hydrochloric acid, hydrobromic acid, phosphoric acid, and the like), organic acid salts (e.g., salts formed using acetic acid, propionic acid, glutamic acid, citric acid, and the like) salts, and quaternary ammonium salts (e.g., salts formed using methyl iodide, ethyl iodide, and the like). Acidic HTR1F antagonists may be contacted with bases to provide base salts such as alkali and alkaline earth metal salts (e.g., sodium, lithium, potassium, calcium, and magnesium salts), as well as ammonium salts (e.g., ammonium, trimethyl-ammonium, diethylammonium, and tris-(hydroxymethyl)-methyl-ammonium salts).

In some embodiments, the neutral forms of the compounds may be regenerated by contacting the salt with a base or acid and isolating the parent compound in the conventional manner if desired. In some embodiments, the parent form of the compound may differ from various salt forms in certain physical properties, such as solubility in polar solvents, but otherwise the salt forms may be equivalent to the parent form of the compound.

As used herein, the term “pharmaceutically acceptable excipient” refers to a substance that aids the administration of an HTR1F antagonist or other compound to a subject. By “pharmaceutically acceptable,” it is meant that the excipient is compatible with the other ingredients of the formulation and is not deleterious to the recipient thereof. It is understood, for example, that pharmaceutically acceptable excipients and salts are non-toxic. Useful pharmaceutical excipients include, but are not limited to, solvents, diluents, pH modifiers, and solubilizers.

As used herein, the terms “treat,” “treatment,” and “treating” refer to any indicia of success in the treatment or amelioration of a condition (e.g., diabetes), injury, pathology, or symptom, including any objective or subjective parameter such as abatement; remission; diminishing of symptoms or making the symptom, injury, pathology or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter; including, e.g., the result of a physical examination.

As used herein the term “effective amount” refers to a dose of a compound such as an HTR1F antagonist that produces the outcome for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition, 2006, Brunton, Ed., McGraw-Hill; and Remington: The Science and Practice of Pharmacy, 21^{st} Edition, 2005, Hendrickson, Ed., Lippincott, Williams & Wilkins).

As used herein, the terms “antagonizing” and “inhibiting” refer to reducing the level of activity (e.g., signaling activity) of target such as HTR1F which can be assessed, for example, using an in vitro assay or other suitable assay. Inhibition of target activity caused by a particular substance (e.g., an HTR1F inhibitor as described herein) can be expressed as the percentage of the activity measured in the absence of the substance under similar conditions. The ability of a particular substance to inhibit a target can be expressed as an IC₅₀ value, i.e., the concentration of the compound required to reduce the activity of the enzyme to 50% of its maximum activity.

As used herein, the term “subject” refers to animals such as mammals, including, but not limited to, primates (e.g., humans), cows, sheep, goats, horses, dogs, cats, rabbits, rats, mice and the like.

The terms “about” and “around,” as used herein to modify a numerical value, indicate a close range surrounding that explicit value. If “X” were the value, “about X” or “around X” would indicate a value from 0.8X to 1.2X, preferably a value from 0.9X to 1.1X, and, more preferably, a value from 0.95X to 1.05X. Any reference to “about X” or “around X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” and “around X” are intended to teach and provide written description support for a claim limitation of, e.g., “0.98X.”

### II. Methods for Treatment of Diabetes

As described herein, it has been discovered that knockdown of human serotonin receptor 1F (HTR1F) improves primary human beta cell survival in the setting of pancreatic islet transplantation. In contrast, HTR1F agonists were found to inhibit human beta cell function and cause human beta cell death. HTR1F antagonists, including those initially contemplated for the treatment of anxiety, can be used to improve primary human beta cell survival and function during islet transplant and in a patient's own beta cells during treatment of diabetes.

Provided herein are methods for treating diabetes. The methods include administering a therapeutically effective amount of a serotonin receptor 1F (HTR1F) antagonist to a subject in need thereof. The subject may have, for example, type 1 diabetes, type 2 diabetes, gestational diabetes, or insulin resistance. In some embodiments, the subject has type 1 diabetes, type 2 diabetes, or gestational diabetes. In some embodiments, the HTR1F antagonist is administered to the subject in conjunction with a pancreatic islet transplantation procedure, as described below.

In some embodiments, the HTR1F antagonist is a substituted piperidine compound according to Formula I

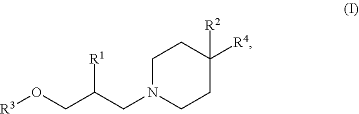

or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

In some embodiments, R¹ and R² are hydroxy. In some embodiments, R³ is naphthyl. In some embodiments, R⁴ is quinolin-3-yl. Compounds of Formula I can be prepared as described, for example, in U.S. Pat. No. 6,242,450.

In some embodiments, the HTR1F antagonist is methiothepin, methysergide, a methysergide derivative, or a pharmaceutically acceptable salt thereof.

In some embodiments, the HTR1F antagonist is methiothepin (CAS Registry No. 20229-30-5; 1-(10,11-dihydro-8-(methylthio)dibenzo(b,f)thiepin-10-yl)-4-methylpiperazine)

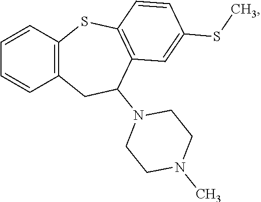 [SMILES: CSc1ccc2c(c1)C(N1CCN(C)CC1)Cc1ccccc1S2]

or a pharmaceutically acceptable salt thereof (e.g., methiothepin maleate, CAS Registry No. 19728-88-2; methiothepin mesylate; or the like).

In some embodiments, the HTR1F antagonist is methysergide (CAS Registry No. 361-37-5; (6aR,9R)—N—((S)-1-hydroxybutan-2-yl)-4,7-dimethyl-4,6,6a,7,8,9-hexahydroindolo[4,3-fg]quinoline-9-carboxamide):

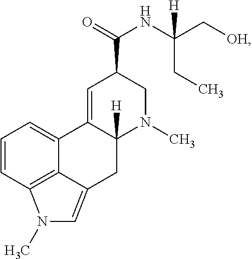

or a pharmaceutically acceptable salt thereof (e.g., methysergide maleate, CAS Registry No. 129-49-7; or the like). In some embodiments, the methysergide derivative is a compound according to Formula II:

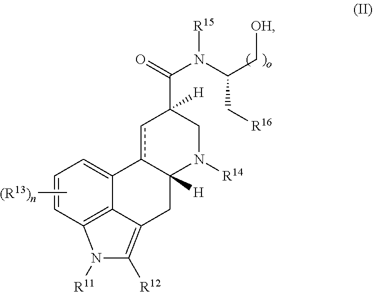

or a pharmaceutically acceptable salt thereof, wherein:
R¹ is selected from the group consisting of hydrogen, optionally substituted C₁₋₄ alkyl (e.g., C₁₋₄ alkyl substituted with one or more fluorine atoms), optionally substituted C₁₋₄ acyl, and optionally substituted C₁₋₄ heteroalkyl;
R¹² is selected from the group consisting of hydrogen, optionally substituted C₁₋₄ alkyl (e.g., C₁₋₄ alkyl substituted with one or more fluorine atoms), optionally substituted C₁₋₄ acyl, halogen, —OH, and optionally substituted C₁₋₄ heteroalkyl;
R¹³ is selected from the group consisting of optionally substituted alkyl, optionally substituted acyl, halo, optionally substituted heteroalkyl, —NO₂, —N₃, —OH, —S(O)ₖR^{13a}, —OR^{13b}, —NR^{13c}R^{13d}, —CONR^{13e}R^{13f}, —CO²R^{13g}, and —O₂CR^{13h};
R¹⁴ is selected from the group consisting of hydrogen and optionally substituted C₁₋₃ alkyl (e.g., C₁₋₃ alkyl substituted with one or more fluorine atoms);
R¹⁵ is selected from the group consisting of hydrogen, optionally substituted C₁₋₄ alkyl (e.g., C₁₋₄ alkyl substituted with one or more fluorine atoms), and optionally substituted C₁₋₄ heteroalkyl;
R¹⁶ is selected from the group consisting of hydrogen, optionally substituted C₁₋₄ alkyl (e.g., C₁₋₄ alkyl substituted with one or more fluorine atoms), and optionally substituted C₁₋₄ heteroalkyl;
subscript n is 0, 1, 2 or 3;
subscript o is 0, 1, 2, 3 or 4;
subscript k is 0, 1 or 2; and
R^{13a}-R^{13h} are independently selected from the group consisting of hydrogen, optionally substituted alkyl, optionally substituted acyl, optionally substituted aryl, optionally substituted arylalkyl, optionally substituted heteroalkyl, optionally substituted heteroaryl, and optionally substituted heteroarylalkyl.

In some embodiments, both R¹¹ and R¹² in compounds of Formula II are not hydrogen. In some embodiments, R¹² in compounds of Formula II is other than hydrogen when R¹¹ and R¹⁴ are methyl, subscript n is 0, subscript o is 1, and R¹⁵ and R¹⁶ are hydrogen. Compounds of Formula II can be prepared as described, for example, in U.S. Pat. No. 8,895,743.

In some embodiments, the HTR1F antagonist may be a compound which is known to antagonize other serotonin receptors and which may also exhibit an inhibitory effect on HTR1F. Methysergide, for example, is known to antagonize HTR1A, HTR2A, HTR2B, HTR2C. Other such non-selective serotonin receptor inhibitors include, but are not limited to, cyanopindolol, ergoline alkaloids (e.g., metergoline, methylergonovine), tricyclic thiepines/thiepanes (e.g., methiothepin), 1-napthylpiperazine, indole alkaloids (e.g., yohimbine), and phenylindoles (e.g., sertindole). Non-selective serotonin receptor inhibitors can be particularly useful for treatment of pancreatic islets prior to a transplant procedure as described herein.

In some embodiments, the HTR1F antagonist is an antibody that binds to HTR1F. As used herein, the term “antibody” refers to a protein with an immunoglobulin fold that specifically binds to an antigen via its variable regions. The term encompasses intact polyclonal antibodies, intact monoclonal antibodies, single chain antibodies, multispecific antibodies such as bispecific antibodies, monospecific antibodies, monovalent antibodies, chimeric antibodies, humanized antibodies, and human antibodies. The term “antibody,” as used herein, also includes antibody fragments that retain antigen-binding specificity via its variable region, including but not limited to Fab, F(ab′)₂, Fv, scFv, and bivalent scFv. Antibodies can contain light chains that are classified as either kappa or lambda. Antibodies can contain heavy chains that are classified as gamma, mu, alpha, delta, or epsilon, which in turn define the immunoglobulin classes, IgG, IgM, IgA, IgD and IgE, respectively. The antibody may be a monoclonal antibody or a polyclonal antibody. A “monoclonal antibody” refers to antibodies produced by a single clone of cells or a single cell line and consisting of or consisting essentially of antibody molecules that are identical in their primary amino acid sequence. A “polyclonal antibody” refers to an antibody obtained from a heterogeneous population of antibodies in which different antibodies in the population bind to different epitopes of an antigen.

Antibodies may be obtained from commercial sources, or may be prepared by immunizing an animal (e.g., a mouse) with an antigen (e.g., an HTR1F protein or fragment thereof) or a mixture of antigens, for the induction of an antibody response. In some embodiments, the antigen or mixture of antigens is administered in conjugation with an adjuvant (e.g., Freund's adjuvant). After an initial immunization, one or more subsequent booster injections of the antigen or antigens may be administered to improve antibody production. Following immunization, antigen-specific B cells are harvested, e.g., from the spleen and/or lymphoid tissue. Phage or yeast display technology can be used to identify antibodies and Fab fragments that specifically bind to selected HTR1F antigens. Alternatively, the genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Antibodies can also be made bispecific, i.e., able to recognize two different antigens. Antibodies can also be heteroconjugates, e.g., two covalently joined antibodies, or immunotoxins. Antibodies can be produced using any number of expression systems, including prokaryotic and eukaryotic expression systems. In some embodiments, the expression system is a mammalian cell expression, such as a hybridoma, or a CHO cell expression system. Many such systems are widely available from commercial suppliers.

In some embodiments, the HTR1F antagonist is a nucleic acid that inhibits the expression of HTR1F. The nucleic acid can be, for example an siRNA or shRNA that mediates RNAi by targeting and inhibiting the expression of HTR1F. Other useful nucleic acid-based inhibitors include microRNAs and antisense oligonucleotides. Relevant sequences and methods for generating inhibitory RNA molecules are known in the art. siRNAs useful for inhibiting the expression of HTR1F are commercially available from, for example, Santa Cruz Biotechnology, Inc. (Catalog No. sc-42229). siRNAs, shRNAs, microRNAs and antisense oligonucleotides, frequently 19-21 nucleotides in length, may also synthesized according to known methods and customized as desired based on the sequence of the target to be inhibited. The nucleic acid may have a single sequence or may be a pool of different sequences (e.g., a pool of 3-7 siRNAs that inhibit the expression of HTR1F). In some embodiments, the nucleic acid comprises a sequence having at least 70% identity (e.g., at least 75, 80, 90, 95, 99, or 100% identity) to SEQ ID NO:1 or SEQ ID NO:2 or a complementary sequence. The terms “identity” and “identical” refer to a sequence having sequence identity to a reference sequence. Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) web site. The BLASTN program for nucleotide sequences uses as defaults a word size (W) of 28, an expectation (E) of 10, M=1, N=−2, and a comparison of both strands. The BLAST algorithm also performs a statistical analysis of the similarity between two sequences (Karlin and Altschul, 1993).

In some embodiments, the HTR1F antagonist is administered as a pharmaceutical composition comprising the HTR1F and a pharmaceutically acceptable excipient.

Liquid pharmaceutical compositions include solutions, suspensions, and emulsions. Such formulations may be administered orally or by injection, that is, intravenously, intramuscularly, intracutaneously, subcutaneously, intraduodenally, or intraperitoneally. Liquid compositions will commonly comprise a solution of the HTR1F antagonist dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are typically sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of HTR1F antagonist in these formulations can vary and will be selected primarily based on fluid volumes, viscosities, body weight, and the like, in accordance with the particular mode of administration selected and the patient's needs.

Oil suspensions can be formulated by suspending an HTR1F antagonist in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. These formulations can be preserved by the addition of an antioxidant such as ascorbic acid. As an example of an injectable oil vehicle, see Minto, J. Pharmacol. Exp. Ther. 281:93-102, 1997. The pharmaceutical compositions can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. Such formulations can also contain a demulcent, a preservative, or a coloring agent.

Suitable compositions for oral administration also include, but are not limited to, tablets, troches, lozenges, dispersible powders or granules, hard or soft capsules, syrups, elixirs, solutions, buccal patches, oral gels, chewing gums, chewable tablets, effervescent powders, and effervescent tablets. Such compositions can contain one or more agents selected from sweetening agents, flavoring agents, coloring agents, antioxidants, and preserving agents in order to provide pharmaceutically elegant and palatable preparations.

Tablets generally contain the active ingredient in admixture with non-toxic pharmaceutically acceptable excipients, including: inert diluents, such as cellulose, silicon dioxide, aluminum oxide, calcium carbonate, sodium carbonate, glucose, mannitol, sorbitol, lactose, calcium phosphate, and sodium phosphate; granulating and disintegrating agents, such as corn starch and alginic acid; binding agents, such as polyvinylpyrrolidone (PVP), cellulose, polyethylene glycol (PEG), starch, gelatin, and acacia; and lubricating agents such as magnesium stearate, stearic acid, and talc. The tablets can be uncoated or coated, enterically or otherwise, by known techniques to delay disintegration and absorption in the gastrointestinal tract and thereby provide a sustained action over a longer period. For example, a time delay material such as glyceryl monostearate or glyceryl distearate can be employed. Tablets can also be coated with a semi-permeable membrane and optional polymeric osmogents according to known techniques to form osmotic pump compositions for controlled release. Hard gelatin capsules can be formulated with the active ingredient is mixed with an inert solid diluent (such as calcium carbonate, calcium phosphate, or kaolin), and soft gelatin capsules can be formulated with the active ingredient mixed with an aqueous medium or an oil medium (such as peanut oil, liquid paraffin, or olive oil).

The HTR1F antagonist is generally administered in amounts sufficient to provide increased beta cell proliferation or beta cell longevity in the subject. For example, the increase in beta cell proliferation upon administration of the HTR1F antagonist may range from about 1% to about 100%, or higher, as compared to the rate of beta cell proliferation observed in absence of the HTR1F antagonist (e.g., in the subject prior to treatment, or in an appropriate control population). The increase in beta cell proliferation can range from about 1% to about 25%, or from about 25% to about 50%, or from about 50% to about 100%, or from about 100% to about 500%. In certain embodiments, the HTR1F antagonist is administered in an amount sufficient to reduce the amount of exogenous insulin, or other agent, necessary for treatment of diabetes in the subject. The decrease in daily dose of exogenous insulin may range, for example, from about 1% to about 25%, or from about 25% to about 50%, or from about 50% to about 100%.

The HTR1F antagonist may be used for treatment of pancreatic islets prior to transplantation in a subject, as described in more detail below. The HTR1F antagonist may also be administered directly to the subject, with or without the transplantation of pancreatic islets. When administered directly to the subject, the HTR1F antagonist will typically be administered at a dose ranging from about 0.1 milligrams to about 1000 milligrams per kilogram of a subject's body weight (i.e., about 0.1-1000 mg/kg). In some embodiments, the HTR1F antagonist is administered at a dose ranging from about 0.1 milligram to about 200 milligrams per kilogram of a subject's body weight (i.e., about 1-100 mg/kg). The dose can be, for example, about 0.1-1000 mg/kg, or about 1-10 mg/kg, or about 10-50 mg/kg, or about 25-50 mg/kg, or about 50-75 mg/kg, or about 75 mg/kg, or about 1-100 mg/kg, or about 1-500 mg/kg, or about 25-250 mg/kg, or about 50-100 mg/kg. The dose can be about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mg/kg. The dosages can be varied depending upon the requirements of the patient, the severity of the diabetes being treated, and the particular formulation being administered. The size of the dose will also be determined by the existence, nature, and extent of any adverse side-effects that accompany the administration of the HTR1F antagonist in a particular patient. Determination of the proper dosage for a particular situation is within the skill of the typical practitioner. The total dosage can be divided and administered in portions over a period of time suitable to treat to the diabetes.

Similarly, administration of the HTR1F antagonist can be conducted for a period of time which will vary depending upon the nature of the diabetes, its severity, and the overall condition of the patient. Administration can be conducted, for example, hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a patient can be monitored for changes in his or her condition and for alleviation of the symptoms of the disorder. The dosage of the HTR1F antagonist can either be increased in the event the patient does not respond significantly to a particular dosage level, or the dose can be decreased if an alleviation of the symptoms is observed, or if unacceptable side effects are seen with a particular dosage. The dosage regimen can consist of two or more different interval sets. For example, a first part of the dosage regimen can be administered to a subject multiple times daily, daily, every other day, or every third day. The dosing regimen can start with dosing the subject every other day, every third day, weekly, biweekly, or monthly. The first part of the dosing regimen can be conducted, for example, for up to 30 days, such as 7, 14, 21, or 30 days. A subsequent second part of the dosing regimen with a different interval administration administered weekly, every 14 days, or monthly can optionally follow, continuing for 4 weeks up to two years or longer, such as 4, 6, 8, 12, 16, 26, 32, 40, 52, 63, 68, 78, or 104 weeks. Alternatively, if the symptoms go into remission or generally improves, the dosage may be maintained or kept at lower than maximum amount. If symptoms worsen, the first dosage regimen can be resumed until an improvement is seen, and the second dosing regimen can be implemented again. This cycle can be repeated multiple times as necessary.

The HTR1F antagonist may be administered with one or more additional active agents. Examples of additional actives include, but are not limited to, metformin, sulfonylureas (e.g., glyburide, glipizide, glimepiride, or the like); meglitinides (e.g., repaglinide, nateglinide, or the like); thiazolidinediones (e.g., rosiglitazone, pioglitazone or the like); DPP-4 inhibitors (e.g., sitagliptin, saxagliptin, linagliptin, or the like); GLP-1 receptor agonists (e.g., exenatide, liraglutide, semaglutide, or the like); and SGLT2 inhibitors (e.g., canagliflozin, dapagliflozin, empagliflozin, or the like). The HTR1F antagonist may also be administered in conjunction with insulin therapy. The insulin therapy may include a short-acting insulin, an intermediate-acting insulin (e.g., NPH insulin), a long-acting insulin (e.g., insulin glargine or insulin detemir), or a combination thereof.

### III. Methods for Transplantation of Pancreatic Islets

Also provided herein are methods for transplanting pancreatic islets. The methods include delivering pancreatic islets to a subject in conjunction with an HTR1F antagonist as described above. The HTR1F antagonist may be, for example, a compound according to Formula I, methiothepin, methysergide, a methysergide derivative (e.g, a compound according to Formula II), or a pharmaceutically acceptable salt thereof. In some embodiments, the HTR1F antagonist is an antibody that binds to HTR1F, or a nucleic acid that inhibits the expression of HT1F. The subject may have type 1 diabetes, type 2 diabetes, gestational diabetes, or insulin resistance

Methods such as the Edmonton Protocol may be used for preparation of pancreatic islets for transplantation. Islet transplantation using the Edmonton Protocol is described By Shapiro, et al. (Transplantation Proceedings, 2001, 33 (7-8): 3502-3503) and Ryan et al. (Diabetes, 2001 (50): 710-719; Diabetes, 2002 (51): 2148-2157). The Edmonton Protocol may include multiple steps, e.g., 7-10 steps, depending on the method employed. The first step involves digestion of pancreatic tissue using one or more enzymes such as collagenases, neutral proteases, and thermolysin. Examples of commercially available enzyme blends suitable for digestion include collagenase NB1/NP (SERVA Electrophoresis GmbH), LIBERASE HI, and LIBERASE MTF C/T (Roche Diagnostics). Digestion can be conducted in a Ricordi chamber or other suitable apparatus, e.g., as described in U.S. Pat. Nos. 6,833,270 and 5,079,160. Following the digestion step, the islets are separated from other cells in the pancreas, e.g., via density gradient centrifugation using a Ficoll, Percoll, or sucrose gradient.

In some embodiments, the pancreatic islets are delivered to the liver of the subject. Separated islets may be introduced into a suitable transplantation medium (e.g., CMRL-1066) and transplanted into the portal vein (e.g., via a percutaneous transhepatic approach). One or more infusions may be used for transfer of the islets to the subject, wherein 5,000-10,000 islet equivalents per kilogram of body weight (or more) are transferred to the subject per infusion. Islet equivalents may be assessed as described for example by Huang et al. (Acta Diabetol, 2013, 50:687-696). The liver is able to regenerate itself when damaged, building new blood vessels and supporting tissue. Therefore, when islets are transplanted into the liver, it is believed that new blood vessels form to support the islets. The insulin that the cells produce is absorbed into the blood stream through these surrounding vessels and distributed through the body to control glucose levels in the blood. Alternatively, pancreatic islets may be implanted under a kidney capsule in the subject.

In some embodiments, pancreatic islets are treated with the HTR1F antagonist before transplantation to the subject. The HTR1F antagonist may be combined, for example, with the pancreatic islets in the transplantation medium prior before transfer to the subject. The amount of the HTRF1 antagonist will depend on factors including, but not limited to, the structure of the particular HTRF1 antagonist employed, the antagonist's affinity for HTRF1 (expressed, for example, as a half maximal inhibitory concentration IC₅₀ or dissociation constant K_{d}), and the number or density of the islets being treated. The HTR1F antagonist may be introduced into cell culture media and/or transplantation media in amounts ranging from a few picomolar to several micromolar. As a non-limiting example, islets may be treated with methysergide or 1-(2-hydroxy-3-(naphthalen-2-yloxy)propyl)-4-(quinolin-3-yl)piperidin-4-ol in amounts ranging from about 10 nM to about 500 nM prior to transplantation. The treatment may be conducted for periods of time ranging from a few minutes to a day or several days. Media containing the HTR1F antagonist may be removed from the islets and replace fresh media prior to the transplantation procedure. Before and/or after the transplantation procedure, the HTR1F antagonist may be administered directly to the subject (e.g., orally or via injection) as described above. The transplantation procedure may also include administration of one or more immunosuppressants to the subject including, but not limited to, corticosteroids, calcineurin inhibitors (e.g., cyclosporine A and tacrolimus), antimetabolites (e.g., azathioprine and mycophenolate mofetil), antilymphocyte antibodies (e.g., muromonab-CD3), and anticytokine receptor antibodies (e.g., daclizumab).

### IV. EXAMPLES

### Example 1. Materials and Methods

The studies described herein were approved by the UCSF Institutional Animal Care and Use Program (AN170193). As human islet donors were anonymized and not available, this was not considered to be human subjects research by the UCSF Institutional Review Board (18-26481). LY344864 and methysergide maleate were obtained from Tocris. Streptozotocin was obtained from Sigma. Pertussis toxin was obtained from Enzo. Statistical tests are listed under each figure legend and were calculated with GraphPad (Prism), R, or custom python script.

Pooled shRNA library construction: A pSicoR-based lentiviral shuttle vector expressing puromycin resistance T2A mCherry under the control of the insulin promoter was a generous gift from Sergio Covarrubius and Michael T. McManus. A custom pool of DNA oligos encoding 12,472 unique shRNAs was synthesized (CustomArray), amplified by PCR (15 cycles, Phusion, NEB) and cloned into the XhoI and EcoRI sites of the vector as previously described[42] with the modification that the original EcoRI site was restored back to the native mir-30 sequence to improve shRNA processing and subsequent knockdown[43].

Human islet transplant screen: Human islets (20-30K IEQ) were isolated from cadaveric donors by the UCSF Human Islet Production Core. Islets were dissociated the day after isolation with collagenase P (Roche) digestion for 15 minutes at 37 degrees, 0.05% trypsin (Thermo) digestion for 10 minutes, and finally gentle trituration to dissociate the islets into single cells. Trypsin was inactivated with fetal bovine serum and the cells were plated on 804G coated plates in CMRL1066 (MediaTech)+B-27 supplement (Thermo), Glutamax (Thermo), non-essential amino acids (Thermo), penicillin and streptomycin. The cells were infected with lentivirus containing the shRNA pool at a MOI of 3-5. Puromycin (3 μg/mL final) was added 3 days after infection and 60% of the surviving cells were transplanted under the kidney capsule of scd-beige (Charles River) mice at day 7-9 after infection while 40% were frozen for later genomic DNA isolation and lentiviral insert sequencing. Four weeks after transplant, the graft was removed and genomic DNA from both samples was isolated. For monitoring of transplant coverage, lentiviral copy number from the pre- and post-transplant samples were measured using digital droplet PCR (QX100, Biorad) using a probe that recognizes the RRE sequence in the lentiviral insert. RRE-F=GGCAAAGAGAAGAGTGGTGC; RRE-R=GACGGTACAGGCCAGACAAT; RRE-probe=CCATAGTGCTTCCTGCTGCTCCC. The lentiviral inserts from the pre-transplant and post-transplant samples were then barcoded, amplified, pooled and sequenced on a HiSeq 4000 (transplants A and B) or MiniSeq (transplant C) (Illumina). Reads were mapped to the original oligo sequences using Hisat2[44]. shRNAs with less than 50 reads were discarded to avoid error due to low counts. Reads counts were then normalized to total read number using DESeq and the ratio of each shRNA post-transplant to pre-transplant was calculated (enrichment score). Gene level p-values were calculated using a custom python script that performed Mann-Whitney U tests comparing the distribution of enrichment scores of all shRNAs to the tested gene to the distribution of the non-targeting shRNAs. These p-values were then FDR corrected with the Benjamini-Hochberg method. Given the anticipated high level of heterogeneity in these different human islets, we set a significance threshold of 0.1.

Human islet donor characteristics: Human islets were obtained from the UCSF Human Islet Production Core or the IsletCore at the University of Alberta.

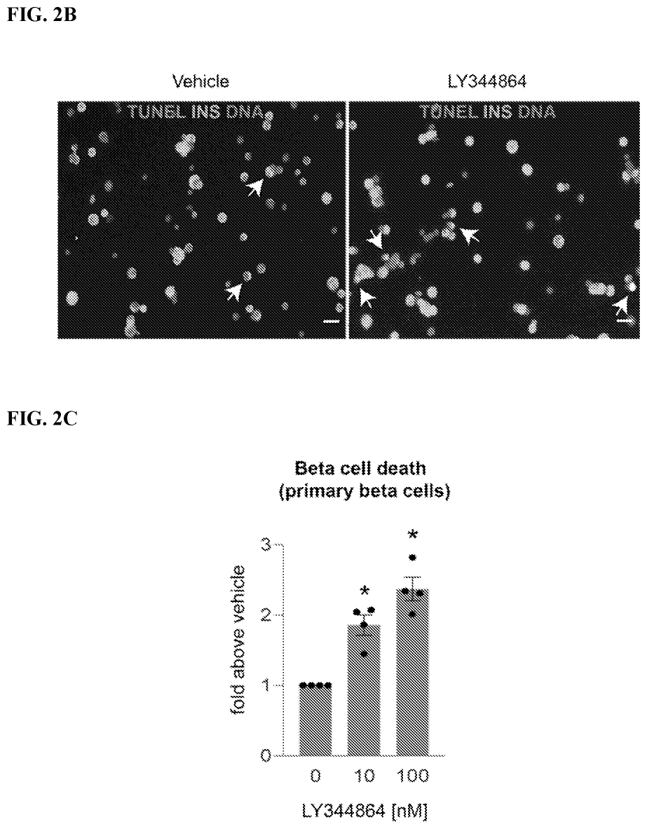

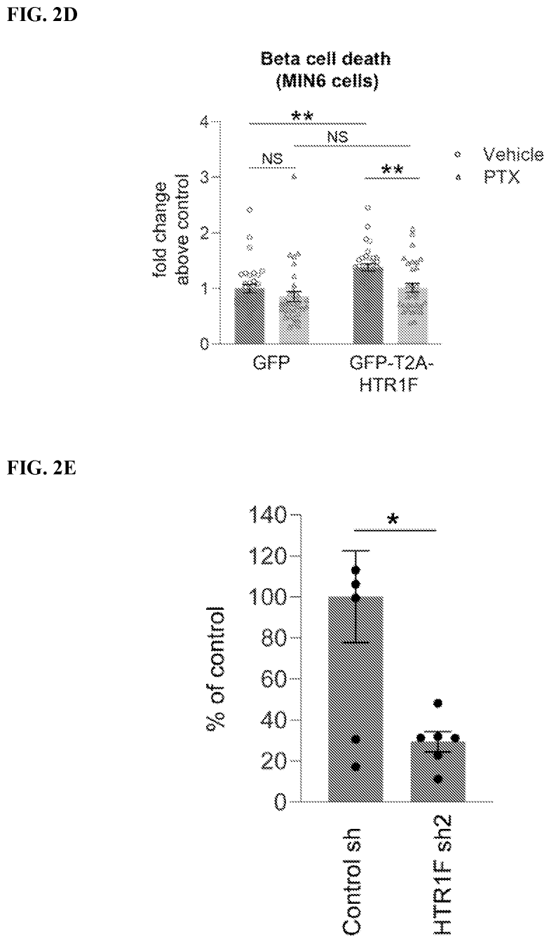

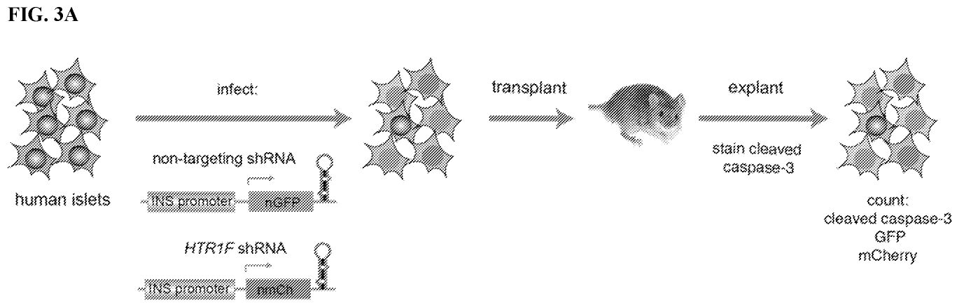

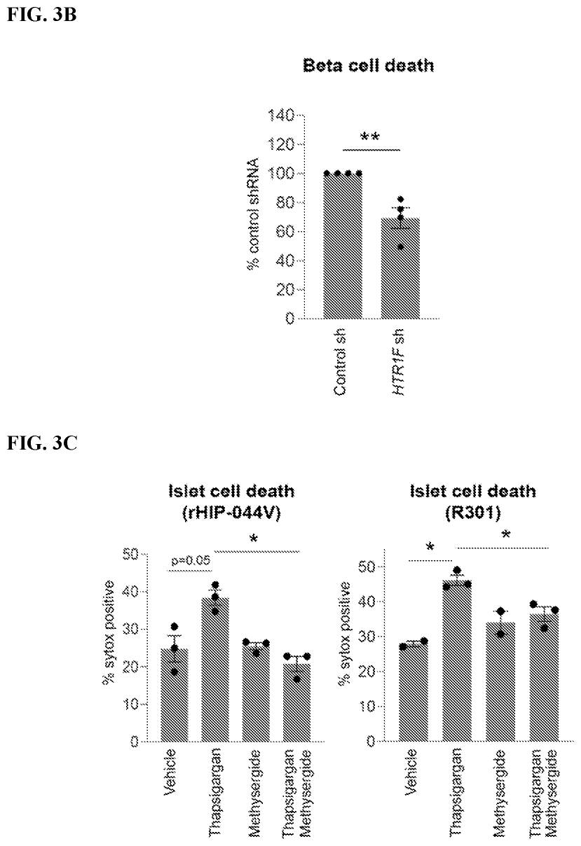

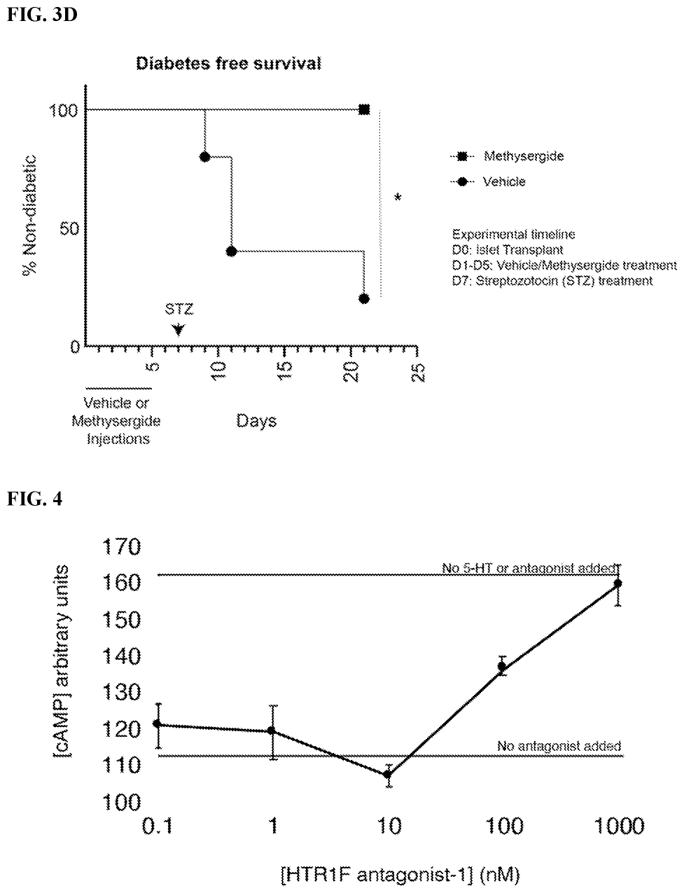

Human beta cell death and proliferation: Islets were dissociated and plated on 804G coated plates as done for the screens. For Sytox Green assays, islets were plated into 96 well plates and Sytox Green (Thermo) was added to the media (30 nM) and green fluorescence and bright field images were captured at 24 hours during culture (Incucyte). The number of green nuclei normalized to the cell density (calculated by brightfield image) was measured in biological triplicate (FIG. 2A). For FIG. 3C, dead nuclei were normalized to total nuclei as measured after treatment of the cells with 0.0625% Triton X-100. For the in vitro TUNEL assay (FIG. 2B and FIG. 2C), cells were fixed in 4% paraformaldehyde in calcium/magnesium free PBS for 15 minutes at room temperature, permeabilized with 0.1% Triton X-100 for 1 hour, blocked with 10% donkey serum in PBS for 1 hour at room temperature, and stained with guinea pig anti-insulin antibody (Agilent) overnight at 4 degrees. Secondary antibody was anti-guinea pig 488 (Thermo) at 1:300 for 1 hour at room temperature. For TUNEL staining, In Situ Death Detection Kit, TMR red (Roche) was used per the manufacturer's protocol in combination with insulin staining as above. Imaging was performed using a Leica SP5 confocal laser microscope. To determine beta cell death after transplant, 5000-6000 IEQs donor islets were infected at an MOI of 10 with lentiviruses containing either a non-targeting shRNA expressed in the 3′UTR of nuclear GFP or an HTR1F shRNA in the 3′ UTR of nuclear mCherry for 5 days.

Non targeting shRNA sequence (including loop):
(SEQ ID NO: 1)
TAAGACTCGAATTGTAGTGTCATAGTGAAGCCACAGATGTATGACACTAC
AATTCGAGTCTTT.
HTRIF targeting shRNA sequence (including loop):
(SEQ ID NO: 2)
TTAGAAGATATACGAAATAATATAGTGAAGCCACAGATGTATATTATTTC
GTATATCTTCTAT

Both were driven by the proximal 360 base pairs of the human insulin promoter. Separate infections were performed to prevent double infection of the beta cells with the two shRNAs. Islets were then pooled and 1000-1500 IEq were then transplanted under the kidney capsule of a scd-beige mouse. The graft was removed after 5 days. Grafts were frozen in O.C.T. (Tissue-Tek) and cryosections were then prepared at 5 μm thickness. Sections were stained as above. The primary antibodies were rabbit anti-cleaved caspase-3 (Cell Signaling; 9661; 1:100), chicken anti-GFP (Aves Labs, 1:100) and mouse anti-mCherry (Clonetech, 1:200) overnight at 4 degrees. Secondary antibodies were anti-rabbit Alexa 647 at 1:300, anti-chicken Alexa 488 at 1:500 and anti-mouse Alexa 555 at 1:300. To determine beta cell proliferation, grafts were stained with anti-Ki67 (BD Pharmigen, 1:150) overnight at 4 degrees. The secondary antibody was anti-mouse Alexa 647 at 1:300.

MIN6 cell death: The human HTR1F cDNA was cloned from 293T cDNA and cloned downstream of a CMV promoter GFP-T2A expression vector with the addition of a viral signal sequence and FLAG epitope tag as previously described[45]. The sequence was confirmed by Sanger sequencing. 10,000 MIN6 cells were plated in a 96-well plate. The following day, the cells were transfected with either the parental GFP-T2A expression vector or GFP-T2A-HTR1F using Lipofectamine 2000 (Thermo Fisher). Six hours after transfection, the media was changed to one containing propidium iodide (Thermo Fisher) at 0.3 ug/mL. The percentage of GFP+ cells that were also PI+ was measured by automated fluorescence microscopy (IncuCyte, Essen Biosciences) at 32-40 hours after transfection. For pertussis toxin (Enzo), cells were treated with 200 ng/mL during cell plating and during transfection.

Minimal human islet transplant model with methysergide: Human islets were treated with 300 nM methysergide maleate or vehicle for 24 hours after isolation. Seven-hundred IEq were then transplanted under the kidney capsule of scd beige mice. Four hours prior to transplant, recipient mice were injected with either 10 mg/kg methysergide maleate or vehicle. After transplant, mice were injected every 24 hours with 10 mg/kg methysergide or vehicle for 5 days. After 48 hours without methysergide treatment to allow washout of the drug, streptozotocin (250 mg/kg) was injected into the peritoneal space with 1 mL of normal saline injected subcutaneously for hydration. 24 hours after streptozotocin injection an additional 1 mL of normal saline was injected subcutaneously with an additional 1 mL into the peritoneal space for hydration. Blood glucose was assessed by glucometer (Freestyle Lite) after a 6 hour fast (9 am-3 pm). Diabetes was defined as a fasting blood sugar >250 or by death at least 3 days after streptozotocin injection.

### Example 2. An In Vivo shRNA Proliferation and Survival Screen in Primary Human Islets

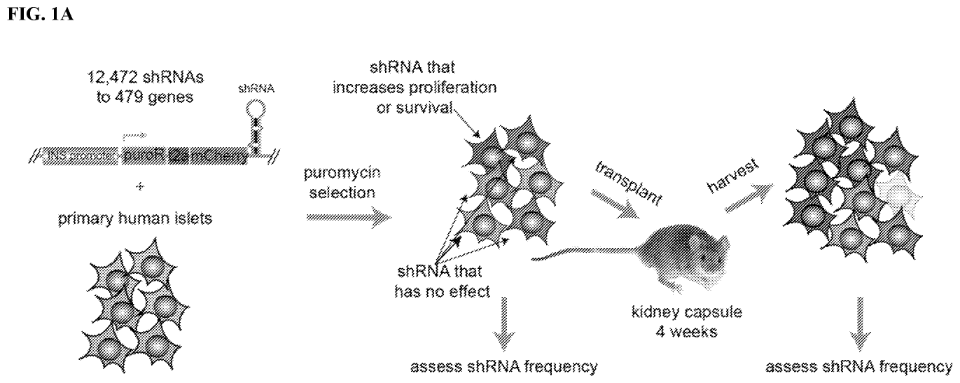

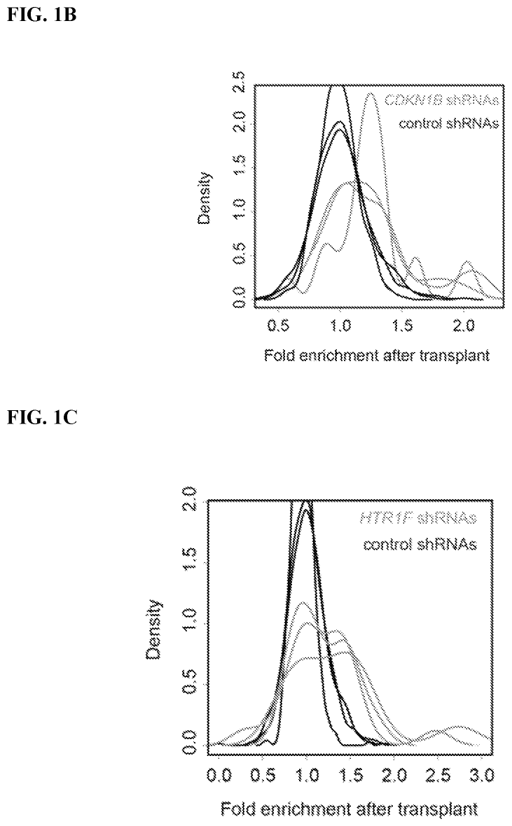

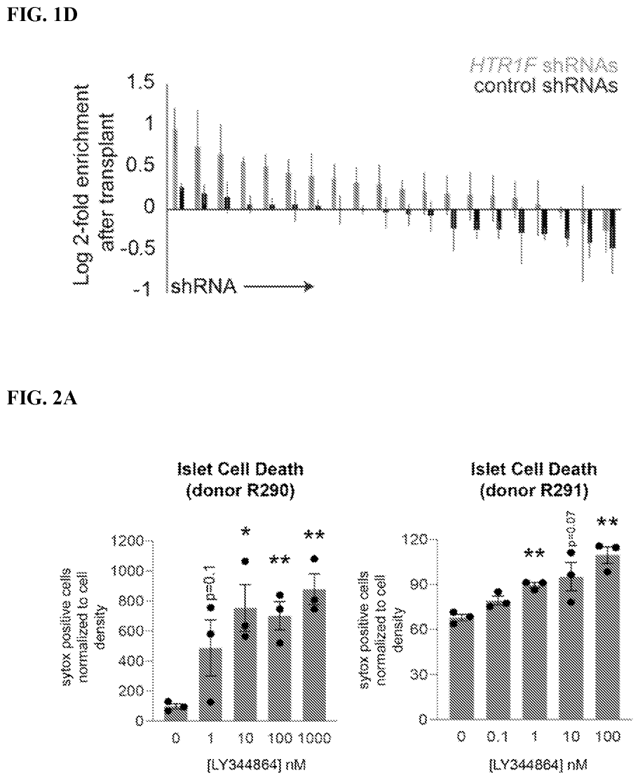

Since a tissue culture dish is not the native environment of the beta cell, in vivo islet transplantation of human beta cells has been reasoned to allow for longer term and more physiologic studies of beta cell proliferation and survival [13]. A pooled shRNA screen was performed in primary human islets transplanted under the kidney capsule of immunocompromised mice. Primary human beta cells were infected with a custom, pooled library of 12,472 independent shRNAs under the control of the insulin promoter (FIG. 1A). A puromycin resistance gene was also expressed under the control of the same promoter to allow for selection of infected beta cells. Each of 479 target genes had 25 independent shRNAs targeting it. Five hundred non-targeting shRNAs were included as negative controls. The targeted genes were chosen based on expression in primary human beta cells and annotation for either druggability or cell surface expression [14, 15]. Genomic DNA was extracted from approximately half of the cells prior to transplant and the remaining half of the cells were transplanted under the kidney capsule of an immunocompromised mouse. Four weeks after transplant, the graft was harvested and genomic DNA was isolated. In order to address this likely small signal to noise ratio and to ensure biological reproducibility, the screen was performed in biological triplicate, using islets from 3 independent donors.

Digital droplet PCR was used to estimate lentiviral copy number from the genomic DNA samples before and after the transplant. Sixty to eighty percent of lentiviral copies were lost after the transplant period, consistent with reported levels of beta cell loss after kidney capsule transplant[3]. After transplant, the cell coverage (i.e., the number of lentiviral inserts recovered per unique shRNA in the original library) ranged between 50-500. The frequency of each shRNA was then measured from the pre-transplant and post-transplant genomic DNA samples by deep sequencing of the integrated lentiviral inserts. A focus on shRNAs that became enriched after transplant was made, as these are likely more specific (i.e., not simply causing increased cell death) and would potentially identify more feasible drug targets (as most therapeutics are inhibitors). A p-value for each gene was calculated based on the enrichments of all shRNAs targeting that gene as compared to those of the 500 non-targeting shRNAs using a multiple testing corrected Mann-Whitney U test. It is noted that the screen that identified the lowest numbers of hits (transplant C) was also the lowest in terms of cell coverage.

If there was beta cell proliferation, enrichments were expected to be modest because not all cells with knockdown of a gene would likely proliferate. In the case of DYRKI1A inhibition, arguably the gold standard to trigger human beta cell replication, only a small fraction of treated cells actually enter the cell cycle[7]. If 50% of cells with knockdown of a gene divide twice, the expected enrichment of that shRNA would be 2-fold. If there was prevention of beta cell death, the maximum enrichment is expected to be inversely proportional to the fraction of cells that die after transplant. In the present case, an enrichment cap was estimated at approximately 5-fold, assuming that knockdown of the gene of interest protects 100% of the cells from death and given 60-80% cell death after transplant. In contrast, dropout screens in cell lines are often capable of generating thousands of fold enrichments or depletions due to ongoing death and proliferation[16].

shRNAs targeting two genes were statistically significantly enriched after transplant in all 3 donors. One gene was CDKN1B or p27Kip1, a cyclin dependent kinase (CDK) inhibitor. Examination of the enrichment ratios for non-targeting shRNAs (FIG. 1B, black lines) revealed a symmetric distribution of enrichment centered around 1 (no enrichment after transplant) while the CDKN1B shRNAs (FIG. 1B, grey lines) had a tail to the right with 1.2-2-fold enrichment after transplant. Notably, there were some shRNAs to CDKN1B that did not become enriched. This was expected since many computationally predicted shRNAs do not knockdown the intended target and thus should not show enrichment. Knockdown or knockout of CDKN1B is known to increase human beta cell proliferation[17] and mouse beta cell proliferation[18], thus validating the approach employed in this study. shRNAs to CDKN2B, another CDK inhibitor, were enriched after transplant in 2 of the 3 donors. Finding these two CDK inhibitors in the screen suggests that the screening method was capable of detecting negative regulators of human beta cell proliferation.

The second gene with statistically significant enriched shRNAs after transplant in all 3 donors was HTR1F (FIG. 1C). The behavior of 20 of the 25 different HTR1F targeting shRNAs also shown—the 5 other HTR1F shRNAs did not have enough reads to reliably quantitate. The same 500 non-targeting shRNAs shown in FIG. 1B are again shown in black. As with CDKN1B, there were a subset of shRNAs to HTR1F that became enriched after transplant in all three donors (FIG. 1C). Importantly, the same HTR1F shRNAs were enriched after transplant in all 3 donors (FIG. 1D).

HTR1F is a class A GPCR whose ligand, serotonin, is released from pancreatic beta cells in a glucose-dependent fashion, suggesting a possible autocrine or paracrine loop[19]. In mice, serotonin plays an important role in beta cell proliferation during pregnancy, lactation, and the perinatal period through the Gαq-coupled Htr2b receptor[20-22]. Serotonin also increases glucose stimulated insulin secretion (GSIS) through HTR2B in adult mice and human islets[23]. In pregnancy, serotonin increases GSIS through Htr3a[24]. HTR1F in the alpha cell negatively regulates glucagon secretion in a paracrine response to serotonin release from beta cells[25], but there is no known role for HTR1F in the beta cell. Previously published mRNA-seq data shows that HTR1F is also expressed in sorted primary human beta cells at 5.3 transcripts per million (TPM) [14, 26], making it the most highly expressed serotonin GPCR by 10-fold in the primary human beta cell. Previously published single cell RNA-seq also confirms that HTR1F is expressed in the human beta cell, alpha cell, delta cell, and gamma cell but not in the acinar cell[27, 28]. Notably, while HTR1F is among the most highly expressed GPCRs in human islets, it is not expressed in the mouse or rat islet[29, 30]-highlighting the importance of screening in human beta cells.

### Example 3. HTR1F Signaling Induces Beta Cell Death

Gαi signaling is also known to restrain beta cell proliferation in mice[31]. However, it was reasoned that since adult human beta cell proliferation is not induced by GLP-1, which triggers Gas signaling[13], knockdown of a Gαi-coupled receptor would be unlikely to cause human beta cell proliferation. Instead, it was hypothesized during the present studies that triggering Gαi signaling might induce cell death. Indeed, the HTR1F specific agonist, LY344864, was found to increase primary human islet cell death in two independent donors (FIG. 2A). To confirm this, the frequency of TUNEL positive cells among insulin expressing cells was measured in an additional 4 donors after LY344864 treatment. LY344864 was found to increase beta cell death at 10 nM, near its reported Ki (6 nM) and EC50 (3 nM)[32](FIGS. 2B and 2C). Importantly, the Ki of LY344864 for the other HTR1 receptors is >500 nM and the Ki for the other serotonin family GPCRs and adrenergic receptors is 1500-5000 nM, more than 50-fold higher than the concentrations where it increases beta cell death[33].

Since mouse beta cells do not express HTR1F[34, 35], human HTR1F was ectopically expressed in the MIN6 mouse beta cell line MIN6. It was found that HTR1F expression alone was sufficient to induce cell death (FIG. 2D). This could reflect basal activity of HTR1F in the setting of over-expression or stimulation of HTR1F by endogenous serotonin production in MIN6 cells[21]. Regardless, these data confirmed that HTR1F signaling induces beta cell death. This cell death was blocked by pre-incubation of the cells with pertussis toxin, suggesting that HTR1F-induced cell death is dependent on Gαi signaling.

### Example 4. HTR1F Knockdown Prevents Human Beta Cell Death

Next, it was asked if HTR1F knockdown could reduce human beta cell death after transplant. One of the enriched shRNAs to HTR1F identified in the screen was cloned and its ability to efficiently reduce HTR1F expression was validated (FIG. 2E). To reduce variability between transplants and between donors, intact primary human islets were infected with either control shRNA or HTR1F shRNA lentiviruses (FIG. 3A) prior to pooling of the infected islets and transplantation of the mixture into a single mouse recipient. To distinguish between the control shRNA expressing beta cells and HTR1F shRNA expressing beta cells, the control shRNA was co-expressed with nuclear GFP and the HTR1F shRNA was co-expressed with nuclear mCherry. The grafts were harvested 4 days after transplant, as most death is believed to occur in the immediate post-transplant period. The frequency of cleaved caspase-3+ cells in the GFP+ population (control knockdown) was measured and compared to the frequency of cleaved caspase-3+ cells in the mCherry+ population (HTR1F knockdown). Over 4 independent donors (and 4 independent transplants), the frequency of cell death in the HTR1F knockdown cells was reduced by 30% as compared to control knockdown (FIG. 3B).

The frequency of Ki67+ positivity in control and HTR1F knockdown cells in these transplants was also measured. Approximately 1000 infected beta cells per knockdown condition for each donor were counted, and only a single Ki67 positive cell with HTR1F knockdown was found. Zero Ki67 positive cells with control knockdown were found. These data suggest that the effects of HTR1F knockdown are likely limited to increasing survival, at least during the first 5 days after transplant.

### Example 5. Methysergide, an HTR1F Antagonist, Prevents Human Beta Cell Death In Vitro and Improves Glycemia in a Marginal Islet Transplant Model

Methysergide is known to potently inhibit HTR1F[33] in addition to other HTR1 and HTR2 class receptors. Since HTR1F is the most highly expressed serotonin GPCR in human beta cells, it was envisioned that methysergide might predominantly block HTR1F and Gαi signaling. Indeed, methysergide is known to increase insulin secretion in humans[36, 37]. While methysergide alone had no effect on islet cell death in dissociated cultures, methysergide was found to prevent death triggered by the ER stress inducer thapsigargan (FIG. 3C).

To ask if improved human beta cell survival due to HTR1F inhibition could improve glycemia after transplant, human islets were treated with methysergide or vehicle and then a marginal mass of islets was transplanted into mice. Recipient mice were treated with daily injections of methysergide or vehicle for an additional 5 days after transplant. After 2 days to allow for methysergide clearance, the endogenous mouse beta cells were destroyed using streptozotocin and monitored for diabetes development. This brief, peri-transplant treatment with methysergide was found to protect mice from the development of diabetes (FIG. 3D). Importantly, this was not an effect of methysergide on the efficiency of mouse beta cell ablation as all mice treated with methysergide developed diabetes after the human islet grafts were surgically removed (data not shown).

In summary, the feasibility of a pooled shRNA screening system to identify regulators of primary human beta cell proliferation and survival has been demonstrated. Using this system, a novel role of HTR1F in beta cell survival has been elucidated. Methysergide, a drug previously used for migraine, was repurposed to improve glycemia in a marginal mass islet transplant model. While methysergide is no longer commonly used due to the risk of retroperitoneal fibrosis during chronic treatment, shorter courses of methysergide (as used here) for recalcitrant migraine prophylaxis minimize this risk and are still occasionally used[38].

Islets infused into the portal vein of human patients experience immediate cell death due to the immediate blood mediated inflammatory reaction (IBMIR)[39, 40]. Notably, a key feature of the IBMIR is platelet activation which would release serotonin immediately around the transplanted islets. Without wishing to be bound by any particular theory, it is believed that part of the IBMIR might be mediated by a serotonin-HTR1F signaling axis. An HTR1F agonist, lasmiditan, has been recently approved for acute migraine treatment[41]. While there are no reports of hyperglycemia in patients using this medication, the data provided herein suggests that it could have negative effects on the beta cell, especially in the setting of transplant or other beta cell stress. Conversely, HTR1F-specific antagonists are expected to be therapeutically useful to improve human islet transplant survival to cure type 1 diabetes patients.

### Example 6. Inhibition of HTR1F in Human Embryonic Kidney Cells

293T cells were transiently transfected with the Glosensor plasmid (Promega) and the HTR1F cDNA. The cells were pre-treated with 1-(2-hydroxy-3-(naphthalen-2-yloxy)propyl)-4-(quinolin-3-yl)piperidin-4-ol at the concentrations shown in FIG. 4 for 10 minutes, then with serotonin (5 nM), and then forskolin and luminescence was determined. n=5, error bars show standard error. Without 5-HT treatment or antagonist treatment, cells had a cAMP level of 162 (red line) as shown in FIG. 4. Without antagonist, cells had a cAMP level of 112 (orange line). N=5 per concentration.

### Example 7. Stimulation of Insulin Production in Human Islets Treated with HTR1F Antagonist

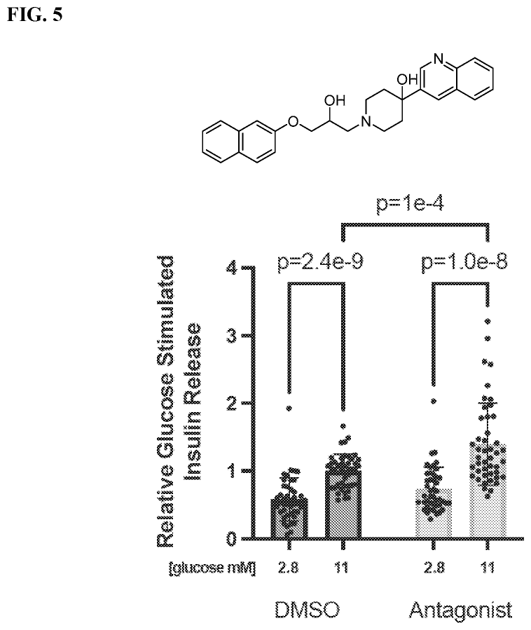

Human islets were rested for 24 hours after receipt in CMRL 1066 (Media Tech) supplemented with penicillin and streptomycin, B27 supplement, Glutamax (Thermo), and non-essential amino acids (Thermo). For both the test treatment group and the control treatment group, 40-60 islets for each of the 5 replicates were hand-picked. The islets were equilibrated for two hours at 37° C. in Kreb-Ringer Bicarbonate HEPES Buffer pH 7.4 (KRBH; 137 mM NaCl, 4.7 mM KCl, 1.2 mM KH₂PO₄, 1.2 mM MgSO₄, 2.5 mM CaCl₂), 25 mM NaHCO₃, 20 mM HEPES, 2.8 mM glucose, and 0.25% BSA). Equilibration buffer was then removed and islets were incubated at 37° C. with KRBH pH 7.4 with 2.8 mM glucose and 0.25% BSA and were treated with DMSO or 150 nM HTR1F antagonist 1-(2-hydroxy-3-(naphthalen-2-yloxy)propyl)-4-(quinolin-3-yl)piperidin-4-ol. After 1 hour, islet secretion was collected and islets were treated with KRBH pH 7.4 containing 11 mM glucose and 0.25% BSA and were treated with DMSO or 150 nM antagonist at 37° C. for another hour. Following incubation, islet secretions were collected and the islets were lysed in RIPA buffer to determine the total islet insulin content. Islet secretions were diluted 2-5 fold for analysis with the Mercodia insulin ELISA kit and lysates were diluted 200-500 fold for analysis. Since insulin secretion is highly variable from individual donors, the data shown in FIG. 5 are normalized to the insulin secretion at high glucose (11 mM) from DMSO treated islets (vehicle). These data are from 9 different donors, with 5 replicates for each donor for DMSO treatment and 5 replicates per donor for HTR1F antagonist treatment.

FDA approved drugs that treat diabetes by increasing insulin secretion in patients (e.g., liraglutide, exenatide, glipizide, glyburide) are known to do so in isolated human islets. In fact, most such drugs were initially tested in vitro on human islets as validation before testing in patients. In the present study, HTR1F antagonist was found to increase insulin secretion at high glucose concentration by 40% over 9 different human islet donors. These data indicate that besides increasing human beta cell survival in the setting of islet transplantation, HTR1F antagonists also increase insulin secretion. Therefore, HTR1F antagonism may be useful for treatment any type of human diabetes even in the absence of islet transplant.

### V. References

1. Vantyghem, M. C., et al., Advances in beta-cell replacement therapy for the treatment of type 1 diabetes. Lancet, 2019. 394(10205): p. 1274-1285.
2. Ryan, E. A., et al., Five-year follow-up after clinical islet transplantation. Diabetes, 2005. 54(7): p. 2060-9.
3. Faleo, G., et al., Mitigating Ischemic Injury of Stem Cell-Derived Insulin-Producing Cells after Transplant. Stem Cell Reports, 2017. 9(3): p. 807-819.
4. Butler, A. E., et al., Beta-cell deficit and increased beta-cell apoptosis in humans with type 2 diabetes. Diabetes, 2003. 52(1): p. 102-10.
5. Baeyens, L., et al., (Re)generating Human Beta Cells: Status, Pitfalls, and Perspectives. Physiol Rev, 2018. 98(3): p. 1143-1167.
6. Stewart, A. F., et al., Human beta-cell proliferation and intracellular signaling: part 3. Diabetes, 2015. 64(6): p. 1872-85.
7. Wang, P., et al., A high-throughput chemical screen reveals that harmine-mediated inhibition of DYRK1A increases human pancreatic beta cell replication. Nat Med, 2015. 21(4): p. 383-8.
8. Dirice, E., et al., Inhibition of DYRK1A Stimulates Human beta-Cell Proliferation. Diabetes, 2016. 65(6): p. 1660-71.
9. Shen, W., et al., Inhibition of DYRK1A and GSK3B induces human beta-cell proliferation. Nat Commun, 2015. 6: p. 8372.
10. Shen, W., et al., Small-molecule inducer of beta cell proliferation identified by high-throughput screening. J Am Chem Soc, 2013. 135(5): p. 1669-72.
11. Abdolazimi, Y., et al., CC-401 Promotes beta-Cell Replication via Pleiotropic Consequences of DYRK1A/B Inhibition. Endocrinology, 2018. 159(9): p. 3143-3157.
12. Annes, J. P., et al., Adenosine kinase inhibition selectively promotes rodent and porcine islet beta-cell replication. Proc Natl Acad Sci USA, 2012. 109(10): p. 3915-20.
13. Dai, C., et al., Age-dependent human beta cell proliferation induced by glucagon-like peptide 1 and calcineurin signaling. J Clin Invest, 2017. 127(10): p. 3835-3844.
14. Nica, A. C., et al., Cell-type, allelic, and genetic signatures in the human pancreatic beta cell transcriptome. Genome Res, 2013. 23(9): p. 1554-62.
15. Mi, H., A. Muruganujan, and P. D. Thomas, PANTHER in 2013: modeling the evolution of gene function, and other gene attributes, in the context of phylogenetic trees. Nucleic Acids Res, 2013. 41(Database issue): p. D377-86.
16. Nagy, T. and M. Kampmann, CRISPulator: a discrete simulation tool for pooled genetic screens. BMC Bioinformatics, 2017. 18(1): p. 347.
17. Stein, J., et al., GSK-3 inactivation or depletion promotes beta-cell replication via down regulation of the CDK inhibitor, p27 (Kip1). Islets, 2011. 3(1): p. 21-34.
18. Uchida, T., et al., Deletion of Cdkn1b ameliorates hyperglycemia by maintaining compensatory hyperinsulinemia in diabetic mice. Nat Med, 2005. 11(2): p. 175-82.
19. Gylfe, E., Association between 5-hydroxytryptamine release and insulin secretion. J Endocrinol, 1978. 78(2): p. 239-48.
20. Kim, H., et al., Serotonin regulates pancreatic beta cell mass during pregnancy. Nat Med, 2010. 16(7): p. 804-8.
21. Moon, J. H., et al., Lactation improves pancreatic beta cell mass and function through serotonin production. Sci Transl Med, 2020. 12(541).
22. Moon, J. H., et al., Serotonin Regulates Adult beta-Cell Mass by Stimulating Perinatal beta-Cell Proliferation. Diabetes, 2020. 69(2): p. 205-214.
23. Bennet, H., et al., Serotonin (5-HT) receptor 2b activation augments glucose-stimulated insulin secretion in human and mouse islets of Langerhans. Diabetologia, 2016. 59(4): p. 744-54.
24. Ohara-Imaizumi, M., et al., Serotonin regulates glucose-stimulated insulin secretion from pancreatic beta cells during pregnancy. Proc Natl Acad Sci USA, 2013. 110(48): p. 19420-5.
25. Almaca, J., et al., Human Beta Cells Produce and Release Serotonin to Inhibit Glucagon Secretion from Alpha Cells. Cell Rep, 2016. 17(12): p. 3281-3291.
26. Fadista, J., et al., Global genomic and transcriptomic analysis of human pancreatic islets reveals novel genes influencing glucose metabolism. Proc Natl Acad Sci USA, 2014. 111(38): p. 13924-9.
27. Lawlor, N., et al., Single-cell transcriptomes identify human islet cell signatures and reveal cell-type-specific expression changes in type 2 diabetes. Genome Res, 2017. 27(2): p. 208-222.
28. Korchynska, S., et al., Life-long impairment of glucose homeostasis upon prenatal exposure to psychostimulants. EMBO J, 2020. 39(1): p. e100882.
29. Amisten, S., et al., A comparative analysis of human and mouse islet G-protein coupled receptor expression. Sci Rep, 2017. 7: p. 46600.
30. Rashid, C. S., et al., Transcriptomic Analysis Reveals Novel Mechanisms Mediating Islet Dysfunction in the Intrauterine Growth-Restricted Rat. Endocrinology, 2018. 159(2): p. 1035-1049.
31. Berger, M., et al., Galphai/o-coupled receptor signaling restricts pancreatic beta-cell expansion. Proc Natl Acad Sci USA, 2015. 112(9): p. 2888-93.
32. Phebus, L. A., et al., Characterization of LY344864 as a pharmacological tool to study 5-HT1F receptors: binding affinities, brain penetration and activity in the neurogenic dural inflammation model of migraine. Life Sci, 1997. 61(21): p. 2117-26.
33. Lovenberg, T. W., et al., Molecular cloning and functional expression of 5-HT1E-like rat and human 5-hydroxytryptamine receptor genes. Proc Natl Acad Sci USA, 1993. 90(6): p. 2184-8.
34. Ku, G. M., et al., Research resource: RNA-Seq reveals unique features of the pancreatic beta-cell transcriptome. Mol Endocrinol, 2012. 26(10): p. 1783-92.
35. DiGruccio, MR., et al., Comprehensive alpha, beta and delta cell transcriptomes reveal that ghrelin selectively activates delta cells and promotes somatostatin release from pancreatic islets. Mol Metab, 2016. 5(7): p. 449-458.
36. Quickel, K. E., Jr., J. M. Feldman, and H. E. Lebovitz, Enhancement of insulin secretion in adult onset diabetics by methysergide maleate: evidence for an endogenous biogenic monoamine mechanism as a factor in the impaired insulin secretion in diabetes mellitus. J Clin Endocrinol Metab, 1971. 33(6): p. 877-81.
37. Baldridge, J. A., et al., Potentiation of tolbutamide-mediated insulin release in adult onset diabetics by methysergide maleate. Diabetes, 1974. 23(1): p. 21-4.
38. Koehler, P. J. and P. C. Tfelt-Hansen, History of methysergide in migraine. Cephalalgia, 2008. 28(11): p. 1126-35.
39. Bennet, W., et al., Incompatibility between human blood and isolated islets of Langerhans: a finding with implications for clinical intraportal islet transplantation? Diabetes, 1999. 48(10): p. 1907-14.
40. Bennet, W., et al., Isolated human islets trigger an instant blood mediated inflammatory reaction: Implications for intraportal islet transplantation as a treatment for patients with type 1 diabetes. Upsala Journal of Medical Sciences, 2000. 105(2): p. 125-133.
41. Kuca, B., et al., Lasmiditan is an effective acute treatment for migraine: A phase 3 randomized study. Neurology, 2018. 91(24): p. e2222-e2232.
42. Bassik, M. C., et al., A systematic mammalian genetic interaction map reveals pathways underlying ricin susceptibility. Cell, 2013. 152(4): p. 909-22.
43. Fellmann, C., et al., An optimized microRNA backbone for effective single-copy RNAi. Cell Rep, 2013. 5(6): p. 1704-13.
44. Pertea, M., et al., Transcript-level expression analysis of RNA-seq experiments with HISAT, StringTie and Ballgown. Nat Protoc, 2016. 11(9): p. 1650-67.
45. Ku, G. M., et al., An siRNA screen in pancreatic beta cells reveals a role for Gpr27 in insulin production. PLoS Genet, 2012. 8(1): p. e1002449.

### VI. EXEMPLARY EMBODIMENTS

Exemplary embodiments provided in accordance with the presently disclosed subject matter include, but are not limited to, the claims and the following embodiments:

1. A method for treating diabetes, the method comprising administering a therapeutically effective amount of a serotonin receptor 1F (HTR1F) antagonist to a subject in need thereof.

2. The method of embodiment 1, wherein the subject has type 1 diabetes, type 2 diabetes, or gestational diabetes.

3. The method of embodiment 1 or embodiment 2, wherein the HTR1F is administered to the subject in conjunction with a pancreatic islet transplantation.

4. The method of embodiment 3, wherein pancreatic islets are treated with the HTR1F antagonist before transplantation to the subject.

5. The method of any one of embodiments 1-4, wherein the HTR1F antagonist is a compound according to Formula I

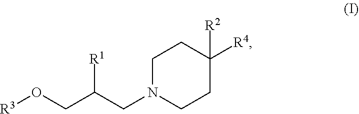

or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

6. The method of embodiment 5, wherein R¹ and R² are hydroxy.

7. The method of embodiment 5 or embodiment 6, wherein R³ is naphthyl.

8. The method of any one of embodiments 5-7, wherein R⁴ is quinolin-3-yl.

9. The method of any one of embodiments 1-4, wherein the HTR1F antagonist is methiothepin, methysergide, or a methysergide derivative.

10. The method of any one of embodiments 1-4, wherein the HTR1F antagonist is an antibody that binds to HTR1F.

11. The method of any one of embodiments 1-4, wherein the HTR1F antagonist is a nucleic acid that inhibits the expression of HTR1F.

12. The method of any one of embodiments 1-11, wherein the HTR1F antagonist is administered as a pharmaceutical composition comprising the HTR1F and a pharmaceutically acceptable excipient.

13. A method for transplanting pancreatic islets to a subject comprising delivering pancreatic islets to the subject in conjunction with an HTR1F antagonist.

14. The method of embodiment 13, wherein the pancreatic islets are delivered to the liver of the subject.

15. The method of embodiment 13, wherein the pancreatic islets are implanted under a kidney capsule in the subject.

16. The method of any one of embodiments 13-15, wherein the pancreatic islets are treated with the HTR1F antagonist before delivering the pancreatic islets to the subject.

17. The method of any one of embodiments 13-16, wherein the HTR1F antagonist is a compound according to Formula I

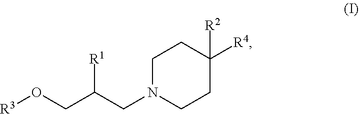

or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

18. The method of embodiment 17, wherein R¹ and R² are hydroxy.

19. The method of embodiment 17 or embodiment 18, wherein R³ is naphthyl.

20. The method of any one of embodiments 17-19, wherein R⁴ is quinolin-3-yl.

21. The method of any one of embodiments 13-16, wherein the HTR1F antagonist is methiothepin, methysergide, or a methysergide derivative.

22. The method of any one of embodiments 13-16, wherein the HTR1F antagonist is an antibody that binds to HTR1F.

23. The method of any one of embodiments 13-16, wherein the HTR1F antagonist is a nucleic acid that inhibits the expression of HT1F.

24. The method of any one of embodiments 13-23, wherein the subject has diabetes.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A method for treating diabetes, the method comprising administering a therapeutically effective amount of a serotonin receptor 1F (HTR1F) antagonist to a subject in need thereof.

2. The method of claim 1, wherein the subject has type 1 diabetes, type 2 diabetes, or gestational diabetes.

3. The method of claim 1, wherein the HTR1F is administered to the subject in conjunction with a pancreatic islet transplantation.

4. The method of claim 3, wherein pancreatic islets are treated with the HTR1F antagonist before transplantation to the subject.

5. The method of claim 1, wherein the HTR1F antagonist is a compound according to Formula I 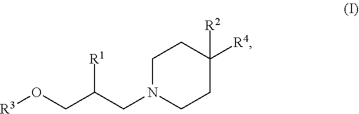
or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

6. The method of claim 5, wherein R¹ and R² are hydroxy.

7. The method of claim 5, wherein R³ is naphthyl.

8. The method of claim 5, wherein R⁴ is quinolin-3-yl.

9. The method of claim 1, wherein the HTR1F antagonist is methiothepin, methysergide, or a methysergide derivative.

10. The method of claim 1, wherein the HTR1F antagonist is an antibody that binds to HTR1F.

11. The method of claim 1, wherein the HTR1F antagonist is a nucleic acid that inhibits the expression of HTR1F.

12. The method of claim 1, wherein the HTR1F antagonist is administered as a pharmaceutical composition comprising the HTR1F and a pharmaceutically acceptable excipient.

13. A method for transplanting pancreatic islets to a subject comprising delivering pancreatic islets to the subject in conjunction with an HTR1F antagonist.

14. The method of claim 13, wherein the pancreatic islets are delivered to the liver of the subject.

15. The method of claim 13, wherein the pancreatic islets are implanted under a kidney capsule in the subject.

16. The method of claim 13, wherein the pancreatic islets are treated with the HTR1F antagonist before delivering the pancreatic islets to the subject.

17. The method of claim 13, wherein the HTR1F antagonist is a compound according to Formula I 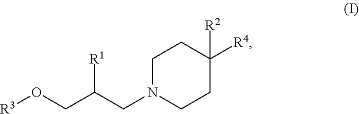
or a pharmaceutically acceptable salt thereof, wherein:
R¹ and R² are independently selected from the group consisting of hydrogen and hydroxy;
R³ is selected from the group consisting of phenyl, naphthyl, quinolinyl, isoquinolinyl, indanyl, 1,2,3,4-tetrahydronaphthyl, indolyl, N—(C₁₋₄ alkyl)indolyl, benzothiazolyl, benzothienyl, benzofuryl, 2,3-dihydrobenzothienyl, 2,3-dihydrobenzofuryl, julolidinyl, and dibenzofuryl;
R³ is optionally substituted with one or two substituents independently selected from the group consisting of C₁₋₆ alkyl, C₁₋₆ acyl, benzoyl, C₁₋₆ alkoxy, phenoxy, C₁₋₆ alkylthio, trifluoromethyl, trifluoromethoxy, and halo; and
R⁴ is selected from the group consisting of pyridin-3-yl, quinolin-3-yl, isoquinolin-4-yl, and quinoxalin-2-yl.

18. The method of claim 17, wherein R¹ and R² are hydroxy.

19. The method of claim 17, wherein R³ is naphthyl.

20. The method of claim 17, wherein R⁴ is quinolin-3-yl.

21. The method of claim 13, wherein the HTR1F antagonist is methiothepin, methysergide, or a methysergide derivative.

22. The method of claim 13, wherein the HTR1F antagonist is an antibody that binds to HTR1F.

23. The method of claim 13, wherein the HTR1F antagonist is a nucleic acid that inhibits the expression of HT1F.

24. The method of claim 13, wherein the subject has diabetes.


SEQUENCE LISTING (5 sequences)

SEQ ID NO: 1 - DNA, 63 bp, Artificial Sequence
  Description of Artificial Sequence Synthetic oligonucleotide
taagactcgaattgtagtgtcatagtgaagccacagatgtatgacactacaattcgagtcttt

SEQ ID NO: 2 - DNA, 63 bp, Artificial Sequence
  Description of Artificial Sequence Synthetic oligonucleotide
ttagaagatatacgaaataatatagtgaagccacagatgtatattatttcgtatatcttctat

SEQ ID NO: 3 - DNA, 20 bp, Artificial Sequence
  Description of Artificial Sequence Synthetic oligonucleotide
ggcaaagagaagagtggtgc

SEQ ID NO: 4 - DNA, 20 bp, Artificial Sequence
  Description of Artificial Sequence Synthetic oligonucleotide
gacggtacaggccagacaat

SEQ ID NO: 5 - DNA, 23 bp, Artificial Sequence
  Description of Artificial Sequence Synthetic oligonucleotide
ccatagtgcttcctgctgctccc